(19) United States
(12) Patent Application Publication
(10) Pub. No.: US 2024/0274240 A1
(43) Pub. Date: Aug. 15, 2024

(54) Method for generating training data based on immunohistochemistry and storage device

(71) Applicant: FUZHOU MAIXIN BIOTECH.CO., LTD., FuZhou (CN)

(72) Inventors: Qinghai YANG, FuZhou (CN); Benliang CHENG, FuZhou (CN); Huiling CHEN, FuZhou (CN); Xiaoya WANG, FuZhou (CN); Nannan WU, FuZhou (CN); Honghui ZHOU, FuZhou (CN)

(21) Appl. No.: 18/598,059

(22) Filed: Mar. 7, 2024

Related U.S. Application Data
(63) Continuation of application No. PCT/CN2022/101122, filed on Jun. 24, 2022.

(30) Foreign Application Priority Data
Sep. 7, 2021 (CN) 202111041413.0

(51) Int. Cl. G16B 40/20; G01N 1/30; G01N 33/574

(52) U.S. Cl. CPC G16B 40/20; G01N 1/30; G01N 33/57415; G01N 2001/302; G01N 2001/307; G01N 2333/4742; G01N 2333/70517; G01N 2474/20

(57) ABSTRACT

The present invention relates to the technical field of computers, and more particularly, to a method for generating training data based on immunohistochemistry, and a storage device. The method for generating the training data based on immunohistochemistry includes the following steps: performing immunohistochemical staining on a target object through different antibodies; labeling the target object according to staining results; and generating training data according to labeling results. According to the method, a pathologist can label a tissue or cell of interest rapidly and conveniently without performing labeling through H&E slicing in combination with histomorphology. In addition, since this tissue or cell is labeled based on an immunohistochemistry technology of a gold standard, this labeling is more accurate compared with manual labeling performed through H&E slicing in combination with histomorphology.

### FIELD OF THE DISCLOSURE

The present invention relates to the technical field of computers, and more particularly, to a method for generating training data based on immunohistochemistry, and a storage device.

### BACKGROUND OF THE INVENTION

At present, all pathology AI software is based on data training for H&E staining and morphology, and requires a large amount of labeling work performed by senior pathologists for H&E stained slices in combination with the morphology to form a dataset for AI learning for the development and application of AI software. This challenges the knowledge and energy of the pathologists, especially in the cases of a large number of stained slices that need to be labeled and inadequate quantity of senior pathologists, will lead to the inability to complete the labeling work in time and affect the subsequent learning progress of the AI software.

### SUMMARY OF THE INVENTION

Therefore, it is necessary to provide a method for generating training data based on immunohistochemistry, which aims to solve the technical problems that all pathology AI software in the prior art is based on data training for H&E staining and morphology and requires the cooperation of senior pathologists, resulting high efficiency, high labor cost, etc. A specific technical solution is as follows: a method for generating training data based on immunohistochemistry includes the following steps:
performing immunohistochemical staining on a target object through different antibodies;
labeling the target object according to staining results; and
generating training data according to labeling results.

Further, the “performing immunohistochemical staining on a target object through different antibodies” specifically further includes the following steps:
adding a CK8/18 primary antibody dropwise to a first slice to be stained for a treated breast cancer tissue, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared AP-Red color developing solution for 8-20 minutes, and rinsing with PBS for 2×3 minutes; and
adding a CK5/6 primary antibody dropwise to the same tissue slice, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared DAB color developing solution for 3-10 minutes, and rinsing with PBS for 2×3 minutes; counterstaining with hematoxylin for 25 seconds, and promoting blue with PBS for 30 seconds; dehydrating in sequence for 3 minutes according to an alcohol gradient of 85%-95%-100%-100%, and finally transparentizing with xylene for 3 minutes; and sealing the slice with neutral balsam.

Further, the “labeling the target object according to staining results” specifically further includes the following steps:
locating the CK5/6 primary antibody in the cytoplasms of basal cells to present a red color; locating the CK8/18 primary antibody in the cytoplasms of normal glandular epithelium or tumor cells of the breast to present a brownish-yellow color; counterstaining the nucleus with hematoxylin to present a blue color; and
distinguishing an in-situ cancer area, an infiltrating cancer area and a micro-infiltrating cancer area according to distribution states of the CK8/18 and CK5/6 antibodies in the breast cancer tissue, and labeling the relevant information.

Further, the “performing immunohistochemical staining on a target object through different antibodies” specifically further includes the following steps:
deparaffinating a slice to be stained for a treated gastric cancer tissue with routine xylene for 3 times, with 6 minutes each time; hydrating with ethanol at a gradient of 100%, 100%, 95% and 85%, with 3 minutes each time, and finally rinsing with tap water; performing antigen retrieval; placing the slice into a wet box, and rinsing with PBS for 3×3 minutes; adding 3% H2O2 dropwise, incubating for 10 minutes, and rinsing with PBS for 3×3 minutes;
spinning the slice to dryness; adding a CD8 antibody dropwise, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared AP-Red color developing solution for 5-15 minutes; counterstaining with hematoxylin for 25 seconds, and promoting blue with PBS for 30 seconds; sealing the slice with an aqueous slice sealing agent;
locating the antibody on a tumor cell membrane and staining in red to acquire a digital pathological image of the tissue slice stained with the CD8 antibody;
soaking the stained tissue slice in xylene, washing the slice sealing agent off, soaking the slice in 95% alcohol, and washing the red stain off;
spinning the tissue slice from which the red stain is washed off to dryness; performing antigen retrieval; adding PD-L1 dropwise, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared DAB color developing solution for 3-10 minutes; counterstaining with hematoxylin for 25 seconds, and promoting blue with PBS for 30 seconds; dehydrating in sequence for 3 minutes according to an alcohol gradient of 85%-95%-100%-100%, finally transparentizing with xylene for 3 minutes; sealing the slice with neutral balsam; and
locating the PD-L1 antibody on cell members of tumor cells as well as cell membranes and cytoplasms of immune cells, and staining in brownish-yellow; and counterstaining the nucleus with hematoxylin to present a blue color, thereby acquiring a digital pathological image of the tissue slice stained with the PD-L1 antibody.

Further, the “labeling the target object according to staining results” specifically further includes the following steps:
performing overlapping comparison on the digital pathological image of the tissue slice stained with the CD8 antibody and the digital pathological image of the tissue slice stained with the PD-L1 antibody, and labeling cells that are positive for both CD8 and PD-L1 in the pathological image after the staining with the PD-L1 antibody.

Further, the “performing immunohistochemical staining on a target object through different antibodies” specifically further includes the following steps:
deparaffinating a slice to be stained for a treated prostate tissue with routine xylene for 3 times, with 6 minutes each time; hydrating with ethanol at a gradient of 100%, 100%, 95% and 85%, with 3 minutes each time, and finally rinsing with tap water; performing antigen retrieval; placing the slice into a wet box, and rinsing with PBS for 3×3 minutes; adding 3% H2O2 dropwise, incubating for 10 minutes, and rinsing with PBS for 3×3 minutes;
spinning the slice to dryness, adding an instant mixed primary antibody dropwise, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a DAB color developing solution for 3-10 minutes, and rinsing with PBS for 2×3 minutes; developing color with an AP-Red color developing solution for 8-20 minutes, and rinsing with running water; counterstaining with hematoxylin for 25 seconds, and promoting blue with PBS for 30 seconds; sealing the slice with neutral balsam; and
acquiring a digital pathological image of the stained tissue slice.

Further, the target object includes one or more of the followings: a target tissue, and a target cell.

Further, the “performing immunohistochemical staining on a target object through different antibodies” specifically further includes the following step:
treating a slice to be stained; and
the “treating the slice to be stained” specifically further includes the following steps:
taking a breast cancer tissue wax block, trimming the wax block and then performing serial slicing with a set thickness of 3 μm; floating the serial slices in cool water to unfold naturally, and then transferring the separated slices to warm water at 45° C. to unfold for 30 seconds; mounting the slices with a glass slice treated with polylysine; baking a prepared tissue chip in a 65° C. oven for 2 hours; taking out and cooling at room temperature; and storing in a −4° C. freezer.

A storage device is configured to store an instruction set therein, the instruction set being configured to: acquire digital pathological images after immunohistochemical staining of a target object with different antibodies;
labeling the target object on the digital pathological images; and
generating training data according to labeling results.

Further, the digital pathological images are acquired in a following manner:
adding a CK8/18 primary antibody dropwise to a first slice to be stained for a treated breast cancer tissue, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared AP-Red color developing solution for 8-20 minutes, and rinsing with PBS for 2×3 minutes; and
adding a CK5/6 primary antibody dropwise to the same tissue slice, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared DAB color developing solution for 3-10 minutes, and rinsing with PBS for 2×3 minutes; counterstaining with hematoxylin for 25 seconds, and promoting blue with PBS for 30 seconds; dehydrating in sequence for 3 minutes according to an alcohol gradient of 85%-95%-100%-100%, and finally transparentizing with xylene for 3 minutes; sealing the slice with neutral balsam;
or
deparaffinating a slice to be stained for a treated gastric cancer tissue with routine xylene for 3 times, with 6 minutes each time; hydrating with ethanol at a gradient of 100%, 100%, 95% and 85%, with 3 minutes each time, and finally rinsing with tap water; performing antigen retrieval; placing the slice into a wet box, and rinsing with PBS for 3×3 minutes; adding 3% H2O2 dropwise, incubating for 10 minutes, and rinsing with PBS for 3×3 minutes;
spinning the slice to dryness; adding a CD8 antibody dropwise, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared AP-Red color developing solution for 5-15 minutes; counterstaining with hematoxylin for 25 seconds, and promoting blue with PBS for 30 seconds; sealing the slice with an aqueous slice sealing agent;
locating the antibody on a tumor cell membrane and staining in red to acquire a digital pathological image of the tissue slice stained with the CD8 antibody;
soaking the stained tissue slice in xylene, washing the slice sealing agent off, soaking the slice in 95% alcohol, and washing the red stain off;
spinning the tissue slice from which the red stain is washed off to dryness, and performing antigen retrieval; adding PD-L1 dropwise and incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared DAB color developing solution for 3-10 minutes; counterstaining with hematoxylin for 25 seconds, and promoting blue with PBS for 30 seconds; dehydrating in sequence for 3 minutes according to an alcohol gradient of 85%-95%-100%-100%, and finally transparentizing with xylene for 3 minutes; sealing the slice with neutral balsam; and
or
deparaffinating a slice to be stained for a treated prostate tissue with routine xylene for 3 times, with 6 minutes each time; hydrating with ethanol at a gradient of 100%, 100%, 95% and 85%, with 3 minutes each time, and finally rinsing with tap water; performing antigen retrieval; placing the slice into a wet box, and rinsing with PBS for 3×3 minutes; adding 3% H2O2 dropwise, incubating for 10 minutes, and rinsing with PBS for 3×3 minutes;
spinning the slice to dryness; adding an instant mixed primary antibody dropwise, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a DAB color developing solution for 3-10 minutes, and rinsing with PBS for 2×3 minutes; developing color with an AP-Red color developing solution for 8-20 min, and rinsing with running water; counterstaining with hematoxylin for 25 seconds, and promoting blue with PBS for 30 seconds; and sealing the slice with neutral balsam.

The present invention has the following beneficial effect: a method for generating the training data based on immunohistochemistry includes the following steps: performing immunohistochemical staining on a target object through different antibodies; labeling the target object according to staining results; and generating training data according to labeling results. According to the method, a pathologist can label a tissue or cell of interest rapidly and conveniently without performing labeling through H&E slicing in combination with histomorphology. In addition, since this tissue or cell is labeled based on an immunohistochemistry technology of a gold standard, this labeling is more accurate compared with manual labeling performed through H&E slicing in combination with histomorphology.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a flowchart of a method for generating training data based on immunohistochemistry in a specific embodiment;

FIG. 2a is a schematic diagram I of a stained breast cancer tissue in a specific embodiment;

FIG. 2b is a schematic diagram II of the stained breast cancer tissue in a specific embodiment;

FIG. 3 is a schematic diagram of a stained gastric cancer tissue in a specific embodiment;

FIG. 4a is a schematic diagram of a normal prostate in a specific embodiment;

FIG. 4b is a schematic diagram I of a stained prostate in a specific embodiment;

FIG. 4c is a schematic diagram II of the stained prostate in a specific embodiment; and

FIG. 5 is a schematic diagram of modules of a storage device in a specific embodiment.

### DESCRIPTION OF THE PREFERRED EMBODIMENTS

In order to describe the technical content, structural characteristics and implementing objects and effects of the technical solutions in detail, the following description is given with reference to the specific embodiments in combination with the accompanying drawings.

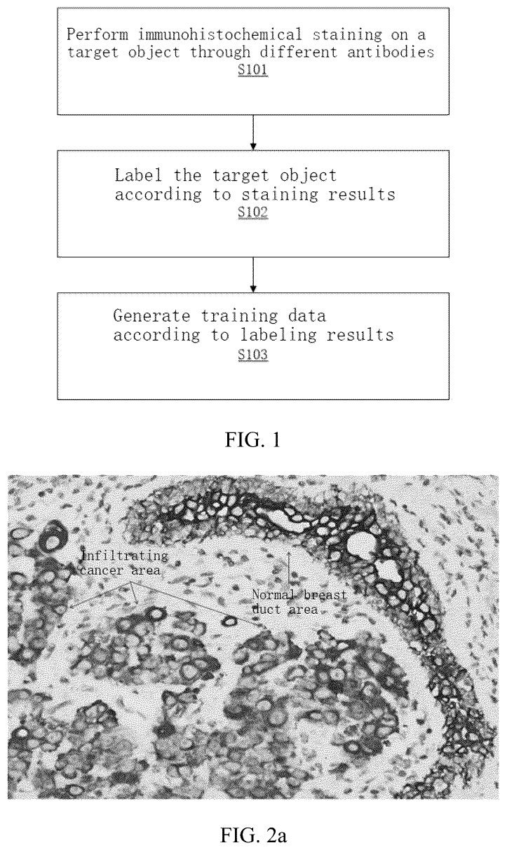

Referring to FIG. 1 to FIG. 4c, in the present embodiment, a method for generating training data based on immunohistochemistry may be applied to a storage device. The storage device includes, but is not limited to, a personal computer, a server, a general-purpose computer, a special-purpose computer, a network device, an embedded device, a programmable device, etc. A specific embodiment includes the following steps:
step S101: performing immunohistochemical staining on a target object through different antibodies;
step S102: labeling the target object according to staining results; and
step S103: generating training data according to labeling results.

According to the method, a pathologist can label a tissue or cell of interest rapidly and conveniently without performing labeling through H&E slicing in combination with histomorphology. In addition, since this tissue or cell is labeled based on an immunohistochemistry technology of a gold standard, this labeling is more accurate compared with manual labeling performed through H&E slicing in combination with histomorphology.

The above method is described with reference to three different embodiments one by one.

### Embodiment 1

The core of Embodiment 1 lies in that: firstly, a specific biomarker of a specific tissue area/cell was subjected to multiple immunohistochemical detection on a slice; a specific area was labeled, followed by immunohistochemical detection of a target biomarker on the same slice; and an interpretation range of the target biomarker was determined.

The following description is made with reference to a breast cancer tissue:
according to the requirements “Guidelines for Chinese Breast Cancer HER2 Detection (in 2019)”, the “Observation Procedure” should be performed prior to interpreting the results of HER2 immunohistochemical detection. That is, the whole slice should be observed under a low-magnification microscope to determine whether the staining was satisfactory and whether there was heterogeneity in HER2 expression. The normal mammary epithelium should not exhibit strong cell membrane staining. The staining of an infiltrating cancer was evaluated only, and the staining of an in-situ cancer cannot be taken as an evaluation object. When the in-situ cancer was accompanied by micro-infiltration, a HER2 state of a micro-infiltrating cancer should be reported if it can be determined in an IHC slice. If there were too few micro-infiltrating lesions in the IHC slice, the HER2 state was hardly accessed and should be noted. Therefore, it was very critical to distinguish an in-situ cancer area, an infiltrating cancer area and a micro-infiltrating cancer area in a breast cancer tissue, in particular a micro-infiltrating cancer area that is difficult to distinguish. The in-situ cancer area, the infiltrating cancer area and the micro-infiltrating cancer area may be distinguished by immunohistochemical staining and labeled for AI learning, so as to achieve the purpose of accurately and quickly distinguishing the in-situ cancer area, the infiltrating cancer area and the micro-infiltrating cancer area based on immunohistochemical staining results by using AI software. In the present embodiment, CK8/18 and CK5/6 antibody reagents were used to perform double staining on the breast cancer tissue, wherein CK8/18 may be used to stain a single layer of epithelial or glandular epithelial cells (red) in a normal or tumor tissue of the breast, while CK5/6 may be used to stain myoepithelial cells (brownish-yellow). The in-situ cancer area, the infiltrating cancer area and the micro-infiltrating cancer area were distinguished according to distribution states of the CK8/18 and CK5/6 antibodies in the breast cancer tissue, and this information was labeled as an AI dataset.

Further, prior to the “performing immunohistochemical staining on a target object through different antibodies”, the method specifically further includes the following steps:
treating a slice to be stained, wherein the breast cancer tissue slice was treated as follows.

### 1. Preparation of Serial Slices of Breast Cancer Tissue

A breast cancer tissue wax block was taken, trimmed and serially sliced with a set thickness of 3 μm; the serial slices were floated in cool water to unfold naturally, and then the separated slices were transferred to warm water at 45° C. to unfold for 30 seconds; the slices were mounted in sequence with a glass slice treated with polylysine; a prepared tissue chip was baked in a 65° C. oven for 2 hours, taken out, cooled at room temperature, and stored in a −4° C. freezer.

### 2. Immunohistochemical Double Staining and Scanning

A breast cancer slice was deparaffinated with routine xylene for 3 times, with 6 minutes each time, hydrated with ethanol at a gradient of 100%, 100%, 95% and 85%, with 3 minutes each time, and finally rinsed with tap water. Antigen retrieval was performed. The slice was placed into a wet box, and rinsed with PBS for 3×3 minutes. The slice was added dropwise with 3% H2O2, incubated for 10 minutes, and rinsed with PBS for 3×3 minutes.

Further, the “performing immunohistochemical staining on a target object through different antibodies” specifically further includes the following steps:
spinning a first slice to be stained for the treated breast cancer tissue to dryness, and adding a CK8/18 primary antibody dropwise to the slice to be stained for the treated breast cancer tissue, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared AP-Red color developing solution for 8-20 minutes, and rinsing with PBS for 2×3 minutes; and
adding a CK5/6 primary antibody dropwise to the same tissue slice, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared DAB color developing solution for 3-10 minutes, and rinsing with PBS for 2×3 minutes; counterstaining with hematoxylin for 25 seconds, and promoting blue with PBS for 30 seconds; dehydrating in sequence for 3 minutes according to an alcohol gradient of 85%-95%-100%-100%, and finally transparentizing with xylene for 3 minutes; and sealing the slice with neutral balsam.
CK5/6 was located in the cytoplasms of basal cells to present a red color, while CK8/18 was located in the cytoplasms of normal glandular epithelium or tumor cells of the breast to present a brownish-yellow color. The nucleus was counterstained with hematoxylin to present a blue color. A digital pathological image was formed by scanning with a tissue slice scanner. In the present embodiment, the room temperature refers to 25° C.

### 3. AI Dataset Labeling of Target Area of Breast Cancer

An in-situ cancer area, an infiltrating cancer area and a micro-infiltrating cancer area were distinguished according to distribution states of the CK8/18 and CK5/6 antibodies in the breast cancer tissue, and this information was labeled as an AI dataset.

Normal glandular epithelium and tumor cells in normal breast tissues and in-situ cancer area of the breast presented red, and periglandular myoepithelium presented brownish-yellow around the periphery of the glandular epithelium, as shown in FIG. 2a and FIG. 2b.

Tumor cells in the infiltrating cancer area presented red as shown in FIG. 2a, and the periglandular myoepithelium was completely absent (i.e., brownish-yellow staining was completely absent).

The AI dataset may be constructed by labeling according to the above immunohistochemical staining information. Then, the HER2 state can be interpreted in a target area required by the above Guidelines in the next serial slice. Details were seen in the following table.

| | | | State of neoplastic glandular |
|---|---|---|---|
| | | | peripheral myoepithelial cell |
| Tissue types | CK5/6 | CK8/18 | (expressing CK5/6) |
| Normal breast | Positive | Positive | Intact |
| Benign | (+) | (+) | |
| epithelial | | | |
| disease | | | |
| in-situ breast | Positive | Positive | Intact or non-intact |
| cancer | (+) | (+) | |
| infiltrating | Negative | Positive | Absent |
| breast cancer | (−) | (+) | |

### Embodiment 2

In addition to the number of PD-L1-positive tumor cells, the number of PD-L1-positive tumor-related immune cells including T lymphocytes (but not including granulocytes, plasma cells and other immune cells) needed to be calculated in PD-L1 interpretation rules for gastric cancer. Therefore, the identification of normal PD-L1-positive T lymphocytes was a key influencing factor in the development of AI software. However, when PD-L1-positive T lymphocytes were labeled only with the help of a H&E stained slice of another slice in a corresponding area on a PD-L1 immunohistochemically stained slice, the pathologist's common sense, energy and other factors can affect the accuracy of labeling. Therefore, in the present embodiment, through multiple staining, the cells that presented both T lymphocyte marker staining and PD-L1-positive staining at the same time in the same slice were PD-L1-positive T lymphocytes that needed to be labeled, and the results were more intuitive and objective.

### 1. Preparation of Gastric Cancer Tissue Slices

A gastric cancer tissue wax block was taken, trimmed and serially sliced with a set thickness of 3 μm; the serial slices were floated in cool water to unfold naturally, and then the separated slices were transferred to warm water at 45° C. to unfold for 30 seconds; the slices were mounted in sequence with a glass slice treated with polylysine; a prepared tissue chip was baked in a 65° C. oven for 2 hours, taken out, cooled at room temperature, and stored in a −4° C. freezer.

### 2. Immunohistochemical Staining and Scanning of T Lymphocyte Marker (CD8)

A sliced was taken, deparaffinated with routine xylene for 3 times, with 6 minutes each time, hydrated with ethanol at a gradient of 100%, 100%, 95% and 85%, with 3 minutes each time, and finally rinsed with tap water. Antigen retrieval was performed. The slice was placed into a wet box, and rinsed with PBS for 3×3 minutes. The slice was added dropwise with 3% H2O2, incubated for 10 minutes, and rinsed with PBS for 3×3 minutes.

The slice was spun to dryness; added with a CD8 antibody reagent diluted at an appropriate proportion dropwise, incubated for 1 hour at room temperature (25° C.), and rinsed with PBS for 3×3 minutes; and added dropwise with a secondary antibody, incubated for 15-30 minutes at room temperature, and rinsed with PBS for 3×3 minutes. PBS was thrown off. Color was developed with a freshly prepared AP-Red color developing solution for 5-15 minutes. Counterstaining was performed with hematoxylin for 25 seconds. Blue was promoted with PBS for 30 seconds. The slice was sealed with an aqueous slice sealing agent. The antibody was located on a tumor cell membrane and stained in red. The tissue slice was scanned with a tissue scanner to form a digital pathological image. The cells with positive cell membranes (red) were T lymphocytes.

### 2.2 Decolorization

The tissue slice was soaked in xylene. The slice sealing agent was washed off. The slice was soaked in 95% alcohol. The red stain was washed off.

### 2.3 PD-L1 Immunohistochemical Staining and Scanning

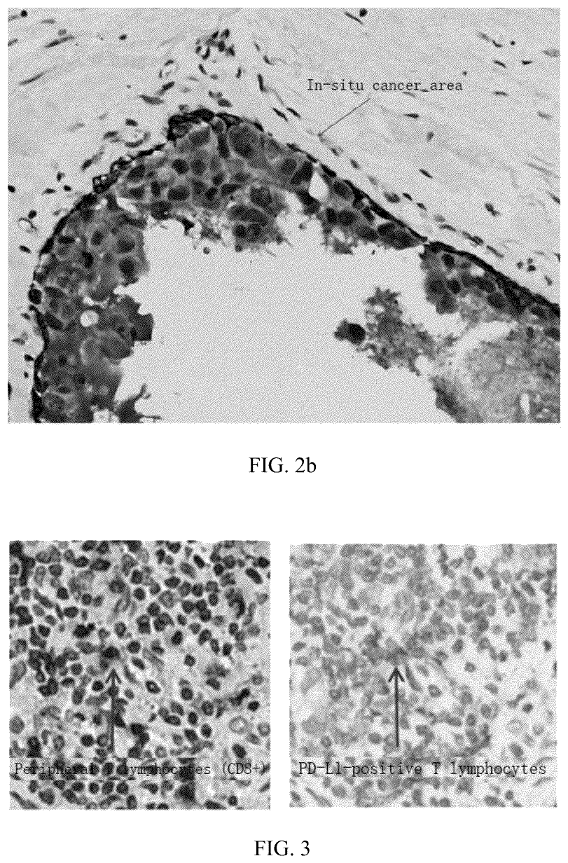

The slice was spun to dryness. Antigen retrieval was performed. The slice was added dropwise with a primary antibody (PD-L1) diluted at an appropriate proportion, incubated for 1 hour at room temperature (25° C.), and rinsed with PBS for 3×3 minutes; and added dropwise with a secondary antibody, incubated for 15-30 minutes at room temperature, and rinsed with PBS for 3×3 minutes. PBS was thrown off. Color was developed with a freshly prepared DAB color developing solution for 3-10 minutes. Counterstaining was performed with hematoxylin for 25 seconds. Blue was promoted with PBS for 30 seconds. The slice was dehydrated in sequence according to an alcohol gradient of 85% (3 minutes)-95% (3 minutes)-100% (3 minutes)-100% (3 minutes), and finally transparentized with xylene for 3 minutes. The slice was sealed with neutral balsam. The PD-L1 antibody was located on cell members of tumor cells as well as cell membranes and cytoplasms of immune cells, and stained in brownish-yellow. The nucleus was counterstained with hematoxylin to present a blue color. A digital pathological image was formed by scanning the tissue slice with a tissue slice scanner, as shown in FIG. 3.

### 3. Labeling of PD-L1-Positive Lymphocytes

Using an image overlapping comparison method, the cells (i.e., PD-L1-positive T lymphocytes) that were positive both for CD8 and PD-L1 were labeled in the PD-L1 immunohistochemically stained slice, for the subsequent construction of the AI dataset.

### Embodiment 3

A combined monoclonal antibody and double enzyme labeling method was used in an immunohistochemical double staining detection kit to detect three antigens, i.e., AMACR/p504s, p63, and CK (HMW), at the same time in one prostate tissue slice. AMACR was highly expressed in prostate cancer, and was not expressed or weakly positive in normal prostate tissues, but can be sporadically expressed in prostatic intraepithelial neoplasia (PIN) and atypical adenomatous hyperplasia (AAH) tissues. The nucleus and cytoplasms of prostatic basal cells were labeled with p63 and CK (HMW), respectively, which can improve the recognition ability of basal cells after the combination of p63 and CK. A normal prostate gland has two layers of cells (basal cells and glandular epithelial cells), while prostate cancer has no or only a small number of basal cells, so various lesions can be directly observed in the same slice by the use of three combined antibodies, which is convenient for comparison and is conducive to the diagnosis and differential diagnosis of prostate cancer, PIN and AAH. In the present embodiment, through the labeling of cells of different colors, the AI software was used to perform AI-assisted interpretation of immunohistochemically double-stained slices of prostate lesion tissues, so as to reduce the workload of pathologists.

### 1. Preparation of Prostate Lesion Tissue Slices

A prostate lesion tissue wax block was taken, trimmed and serially sliced with a set thickness of 3 μm. The serial slices were floated in cool water to unfold naturally, and then the separated slices were transferred to warm water at 45° C. to unfold for 30 seconds. The slices were mounted in sequence with a glass slice treated with polylysine. A prepared tissue chip was baked in a 65° C. oven for 2 hours, taken out, cooled at room temperature, and stored in a −4° C. freezer.

### 2. Immunohistochemical Double Staining and Scanning of Prostate Lesion Tissues

A sliced was taken, deparaffinated with routine xylene for 3 times, with 6 minutes each time, hydrated with ethanol at a gradient of 100%, 100%, 95% and 85%, with 3 minutes each time, and finally rinsed with tap water. Antigen retrieval was performed. The slice was placed into a wet box, and rinsed with PBS for 3×3 minutes. The slice was added dropwise with 3% H2O2, incubated for 10 minutes, and rinsed with PBS for 3×3 minutes.

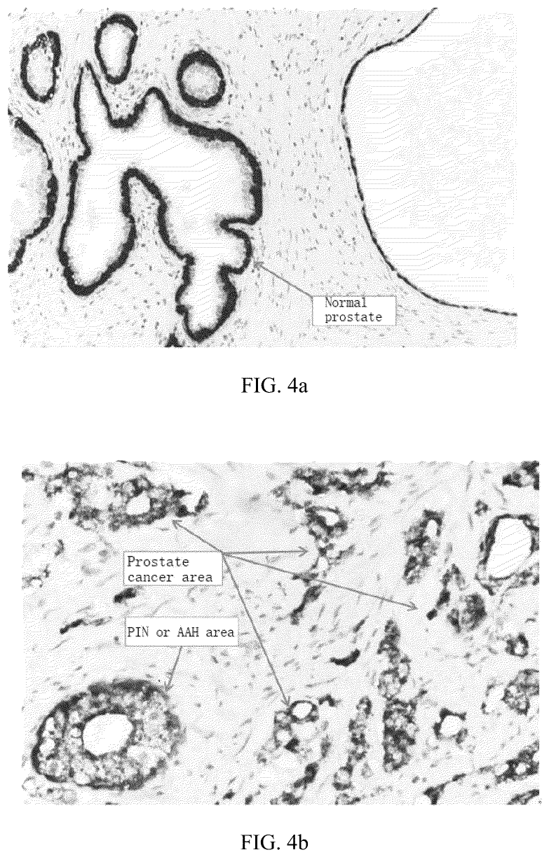

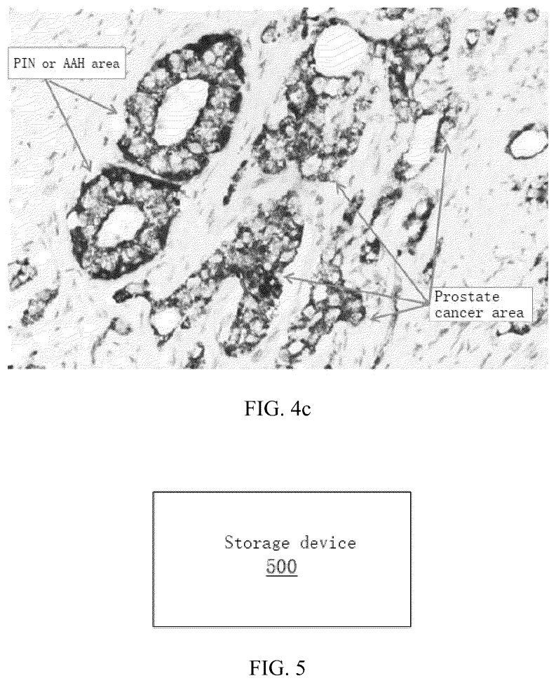

The slice was spun to dryness; added dropwise with an instant mixed primary antibody, incubated for 1 hour at room temperature (25° C.), and rinsed with PBS for 3×3 minutes; and added dropwise with a secondary antibody, incubated for 15-30 minutes at room temperature, and rinsed with PBS for 3×3 minutes. PBS was thrown off. Color was developed with a DAB color developing solution for 3-10 minutes. The slice was rinsed with PBS for 2×3 minutes. Color was developed with an AP-Red color developing solution for 8-20 minutes. The slice was rinsed with running water. Counterstaining was performed with hematoxylin for 25 seconds. Blue was promoted with PBS for 30 seconds. The slice was mounted with neutral balsam. forming a digital pathological image by scanning with a tissue slice scanner. as shown in FIG. 4a, FIG. 4b and FIG. 4c.

### Labeling for Different Expression Models

According to different localizations of different specific biomarkers on a tissues or cells, specific types of tissues or cells were labeled on the same tissue slice, and labeling models for prostate lesion tissues could be divided into three types, as shown in the table below.

Table Classification of labeling models for prostate lesion tissues

| | AMACR/ | | CK(HMW) | |
|---|---|---|---|---|
| | p504s | P63 | or CK5/6 | Feature points |
| Normal prostate tissue | − | + | + | Periglandular basal cells |
| | | | | were intact |
| Prostatic intraepithelial | + | + | + | Periglandular basal cells |
| neoplasia (PIN), atypical | (Brownish- | (Red) | (Red) | were intact or non-intact |
| adenomatous hyperplasia (AAH) | yellow/brown) | | | |
| Prostate cancer (PC) | + | − | − | Basal cells were not |
| | (Brownish- | (Red) | (Red) | seen around the gland |
| | yellow/brown) | | | |

A method for generating training data based on immunohistochemistry includes the following steps: performing immunohistochemical staining on a target object through different antibodies; labeling the target object according to staining results; and generating training data according to labeling results. According to the method, a pathologist can label a tissue or cell of interest rapidly and conveniently without performing labeling through H&E slicing in combination with histomorphology. In addition, since this tissue or cell is labeled based on an immunohistochemistry technology of a gold standard, this labeling is more accurate compared with manual labeling performed through H&E slicing in combination with histomorphology.

Referring to FIG. 5, in the present embodiment, the specific embodiment of a storage device 500 is as follows:
a storage device 500 is configured to store an instruction set therein, the instruction set being configured to: acquire digital pathological images after immunohistochemical staining of a target object with different antibodies;
labeling the target object on the digital pathological images; and
generating training data according to labeling results.

Further, the digital pathological images were acquired in a following manner:
adding a CK8/18 primary antibody dropwise to a first slice to be stained for a treated breast cancer tissue, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared AP-Red color developing solution for 8-20 minutes, and rinsing with PBS for 2×3 minutes; and
adding a CK5/6 primary antibody dropwise to the same tissue slice, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared DAB color developing solution for 3-10 minutes, and rinsing with PBS for 2×3 minutes; counterstaining with hematoxylin for 25 seconds, and promoting blue with PBS for 30 seconds; dehydrating in sequence for 3 minutes according to an alcohol gradient of 85%-95%-100%-100%, and finally transparentizing with xylene for 3 minutes; sealing the slice with neutral balsam;
or
deparaffinating a slice to be stained for a treated gastric cancer tissue with routine xylene for 3 times, with 6 minutes each time; hydrating with ethanol at a gradient of 100%, 100%, 95% and 85%, with 3 minutes each time, and finally rinsing with tap water; performing antigen retrieval; placing the slice into a wet box, and rinsing with PBS for 3×3 minutes; adding 3% H2O2 dropwise and incubating for 10 minutes, and rinsing with PBS for 3×3 minutes;
spinning the slice to dryness; adding a CD8 antibody dropwise, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared AP-Red color developing solution for 5-15 minutes; counterstaining with hematoxylin for 25 seconds, and promoting blue with PBS for 30 seconds; sealing the slice with an aqueous slice sealing agent;
locating the antibody on a tumor cell membrane and staining in red to acquire a digital pathological image of the tissue slice stained with the CD8 antibody;
soaking the stained tissue slice in xylene, washing the slice sealing agent off, soaking the slice in 95% alcohol, and washing the red stain off;
throwing the tissue slice from which the red stain was removed to dryness; performing antigen retrieval; adding PD-L1 dropwise, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared DAB color developing solution for 3-10 minutes; counterstaining with hematoxylin for 25 seconds, and promoting blue with PBS for 30 seconds; dehydrating in sequence for 3 minutes according to an alcohol gradient of 85%-95%-100%-100%, and finally transparentizing with xylene for 3 minutes; sealing the slice with neutral balsam;
or
deparaffinating a slice to be stained for a treated prostate tissue with routine xylene for 3 times, with 6 minutes each time; hydrating with ethanol at a gradient of 100%, 100%, 95% and 85%, with 3 minutes each time, and finally rinsing with tap water; performing antigen retrieval; placing the slice into a wet box, and rinsing with PBS for 3×3 minutes; adding 3% H2O2 dropwise and incubating for 10 minutes, and rinsing with PBS for 3×3 minutes;
spinning the slice to dryness; adding an instant mixed primary antibody dropwise, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a DAB color developing solution for 3-10 minutes, and rinsing with PBS for 2×3 minutes; developing color with an AP-Red color developing solution for 8-20 min, and rinsing with running water; counterstaining with hematoxylin for 25 seconds, and promoting blue with PBS for 30 seconds; and sealing the slice with neutral balsam.

Through the corresponding steps performed by the instruction set of the storage device 500, a pathologist can label a tissue or cell of interest rapidly and conveniently without performing labeling through H&E slicing in combination with histomorphology. In addition, since this tissue or cell is labeled based on an immunohistochemistry technology of a gold standard, this labeling is more accurate compared with manual labeling performed through H&E slicing in combination with histomorphology.

The present invention has the following beneficial effect: a method for generating the training data based on immunohistochemistry includes the following steps: performing immunohistochemical staining on a target object through different antibodies; labeling the target object according to staining results; and generating training data according to labeling results. According to the method, a pathologist can label a tissue or cell of interest rapidly and conveniently without performing labeling through H&E slicing in combination with histomorphology. In addition, since this tissue or cell is labeled based on an immunohistochemistry technology of a gold standard, this labeling is more accurate compared with manual labeling performed through H&E slicing in combination with histomorphology.

It should be noted that, although the above embodiments have been described herein, the patent protection scope of the present invention is not limited accordingly. Therefore, based on the innovative concept of the present invention, the changes and modifications made to the embodiments described herein, or the transformation of the equivalent structures or equivalent processes made by using the contents of the description and drawings of the present invention, or direct or indirect application of the above technical solutions to other related technical fields, are all included in the patent protection scope of the present invention.

What is claimed is:

1. A method for generating training data based on immunohistochemistry includes the following steps:
performing immunohistochemical staining on a target object through different antibodies;
labeling the target object according to staining results; and
generating training data according to labeling results.

2. The method for generating training data based on immunohistochemistry of claim 1, wherein the “performing immunohistochemical staining on a target object through different antibodies” specifically further includes the following steps:
adding a CK8/18 primary antibody dropwise to a first slice to be stained for a treated breast cancer tissue, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared AP-Red color developing solution for 8-20 minutes, and rinsing with PBS for 2×3 minutes; and
adding a CK5/6 primary antibody dropwise to the same tissue slice, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared DAB color developing solution for 3-10 minutes, and rinsing with PBS for 2×3 minutes; counterstaining with hematoxylin for 25 seconds, and promoting blue with PBS for 30 seconds; dehydrating in sequence for 3 minutes according to an alcohol gradient of 85%-95%-100%-100%, and finally transparentizing with xylene for 3 minutes; and sealing the slice with neutral balsam.

3. The method for generating training data based on immunohistochemistry of claim 2, wherein the “labeling the target object according to staining results” specifically further includes the following steps:
locating the CK5/6 primary antibody in the cytoplasms of basal cells to present a red color; locating the CK8/18 primary antibody in the cytoplasms of normal glandular epithelium or tumor cells of the breast to present a brownish-yellow color; counterstaining the nucleus with hematoxylin to present a blue color; and
distinguishing an in-situ cancer area, an infiltrating cancer area and a micro-infiltrating cancer area according to distribution states of the CK8/18 and CK5/6 antibodies in the breast cancer tissue, and labeling the relevant information.

4. The method for generating training data based on immunohistochemistry of claim 1, wherein the “performing immunohistochemical staining on a target object through different antibodies” specifically further includes the following steps:
deparaffinating a slice to be stained for a treated gastric cancer tissue with routine xylene for 3 times, with 6 minutes each time; hydrating with ethanol at a gradient of 100%, 100%, 95% and 85%, with 3 minutes each time, and finally rinsing with tap water; performing antigen retrieval; placing the slice into a wet box, and rinsing with PBS for 3×3 minutes; adding 3% H2O2 dropwise, incubating for 10 minutes, and rinsing with PBS for 3×3 minutes;
spinning the slice to dryness; adding a CD8 antibody dropwise, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared AP-Red color developing solution for 5-15 minutes; counterstaining with hematoxylin for 25 seconds, and promoting blue with PBS for 30 seconds; sealing the slice with an aqueous slice sealing agent;
locating the antibody on a tumor cell membrane and staining in red to acquire a digital pathological image of the tissue slice stained with the CD8 antibody;
soaking the stained tissue slice in xylene, washing the slice sealing agent off, soaking the slice in 95% alcohol, and washing the red stain off;
spinning the tissue slice from which the red stain is washed off to dryness; performing antigen retrieval; adding PD-L1 dropwise, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared DAB color developing solution for 3-10 minutes; counterstaining with hematoxylin for 25 seconds, and promoting blue with PBS for 30 seconds; dehydrating in sequence for 3 minutes according to an alcohol gradient of 85%-95%-100%-100%, finally transparentizing with xylene for 3 minutes; sealing the slice with neutral balsam; and
locating the PD-L1 antibody on cell members of tumor cells as well as cell membranes and cytoplasms of immune cells, and staining in brownish-yellow; and counterstaining the nucleus with hematoxylin to present a blue color, thereby acquiring a digital pathological image of the tissue slice stained with the PD-L1 antibody.

5. The method for generating training data based on immunohistochemistry of claim 4, wherein the “labeling the target object according to staining results” specifically further includes the following steps:
performing overlapping comparison on the digital pathological image of the tissue slice stained with the CD8 antibody and the digital pathological image of the tissue slice stained with the PD-L1 antibody, and labeling cells that are positive for both CD8 and PD-L1 in the pathological image after the staining with the PD-L1 antibody.

6. The method for generating training data based on immunohistochemistry of claim 1, wherein the “performing immunohistochemical staining on a target object through different antibodies” specifically further includes the following steps:
deparaffinating a slice to be stained for a treated prostate tissue with routine xylene for 3 times, with 6 minutes each time; hydrating with ethanol at a gradient of 100%, 100%, 95% and 85%, with 3 minutes each time, and finally rinsing with tap water; performing antigen retrieval; placing the slice into a wet box, and rinsing with PBS for 3×3 minutes; adding 3% H2O2 dropwise, incubating for 10 minutes, and rinsing with PBS for 3×3 minutes;
spinning the slice to dryness, adding an instant mixed primary antibody dropwise, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a DAB color developing solution for 3-10 minutes, and rinsing with PBS for 2×3 minutes; developing color with an AP-Red color developing solution for 8-20 minutes, and rinsing with running water; counterstaining with hematoxylin for 25 seconds, and promoting blue with PBS for 30 seconds; sealing the slice with neutral balsam; and acquiring a digital pathological image of the stained tissue slice.

7. The method for generating training data based on immunohistochemistry of claim 1, wherein the target object includes one or more of the followings: a target tissue, and a target cell.

8. The method for generating training data based on immunohistochemistry of claim 1, the “performing immunohistochemical staining on a target object through different antibodies” specifically further includes the following step:
treating a slice to be stained; and the “treating the slice to be stained” specifically further includes the following steps:
taking a breast cancer tissue wax block, trimming the wax block and then performing serial slicing with a set thickness of 3 μm; floating the serial slices in cool water to unfold naturally, and then transferring the separated slices to warm water at 45° C. to unfold for 30 seconds; mounting the slices with a glass slice treated with polylysine; baking a prepared tissue chip in a 65° C. oven for 2 hours; taking out and cooling at room temperature; and storing in a −4° C. freezer.

9. A storage device is configured to store an instruction set therein, the instruction set being configured to: acquire digital pathological images after immunohistochemical staining of a target object with different antibodies;
labeling the target object on the digital pathological images; and generating training data according to labeling results.

10. The storage device of claim 9, wherein the digital pathological images are acquired in a following manner:
adding a CK8/18 primary antibody dropwise to a first slice to be stained for a treated breast cancer tissue, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared AP-Red color developing solution for 8-20 minutes, and rinsing with PBS for 2×3 minutes; and
adding a CK5/6 primary antibody dropwise to the same tissue slice, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared DAB color developing solution for 3-10 minutes, and rinsing with PBS for 2×3 minutes; counterstaining with hematoxylin for 25 seconds, and promoting blue with PBS for 30 seconds; dehydrating in sequence for 3 minutes according to an alcohol gradient of 85%-95%-100%-100%, and finally transparentizing with xylene for 3 minutes; sealing the slice with neutral balsam.

11. The storage device of claim 9, wherein the digital pathological images are acquired in a following manner:
deparaffinating a slice to be stained for a treated gastric cancer tissue with routine xylene for 3 times, with 6 minutes each time; hydrating with ethanol at a gradient of 100%, 100%, 95% and 85%, with 3 minutes each time, and finally rinsing with tap water; performing antigen retrieval; placing the slice into a wet box, and rinsing with PBS for 3×3 minutes; adding 3% H2O2 dropwise, incubating for 10 minutes, and rinsing with PBS for 3×3 minutes;
spinning the slice to dryness; adding a CD8 antibody dropwise, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared AP-Red color developing solution for 5-15 minutes; counterstaining with hematoxylin for 25 seconds, and promoting blue with PBS for 30 seconds; sealing the slice with an aqueous slice sealing agent;
locating the antibody on a tumor cell membrane and staining in red to acquire a digital pathological image of the tissue slice stained with the CD8 antibody;
soaking the stained tissue slice in xylene, washing the slice sealing agent off, soaking the slice in 95% alcohol, and washing the red stain off; and
spinning the tissue slice from which the red stain is washed off to dryness, and performing antigen retrieval; adding PD-L1 dropwise and incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a freshly prepared DAB color developing solution for 3-10 minutes; counterstaining with hematoxylin for 25 seconds, and promoting blue with PBS for 30 seconds; dehydrating in sequence for 3 minutes according to an alcohol gradient of 85%-95%-100%-100%, and finally transparentizing with xylene for 3 minutes; sealing the slice with neutral balsam.

12. The storage device of claim 9, wherein the digital pathological images are acquired in a following manner:
deparaffinating a slice to be stained for a treated prostate tissue with routine xylene for 3 times, with 6 minutes each time; hydrating with ethanol at a gradient of 100%, 100%, 95% and 85%, with 3 minutes each time, and finally rinsing with tap water; performing antigen retrieval; placing the slice into a wet box, and rinsing with PBS for 3×3 minutes; adding 3% H2O2 dropwise, incubating for 10 minutes, and rinsing with PBS for 3×3 minutes;
spinning the slice to dryness; adding an instant mixed primary antibody dropwise, incubating for 1 hour at room temperature, and rinsing with PBS for 3×3 minutes; adding a secondary antibody dropwise, incubating for 15-30 minutes at room temperature, and rinsing with PBS for 3×3 minutes; throwing off PBS; developing color with a DAB color developing solution for 3-10 minutes, and rinsing with PBS for 2×3 minutes; developing color with an AP-Red color developing solution for 8-20 min, and rinsing with running water; counterstaining with hematoxylin for 25 seconds, and promoting blue with PBS for 30 seconds; and sealing the slice with neutral balsam.
